(19) United States
(12) Patent Application Publication
(10) Pub. No.: US 2020/0306255 A1
(43) Pub. Date: Oct. 1, 2020

(54) COMPOSITIONS AND METHODS FOR TREATING NIEMANN PICK C DISEASE

(71) Applicant: AI Therapeutics, Inc., Guilford, CT (US)

(72) Inventors: Chris Conrad, Guilford, CT (US); Jonathan M. Rothberg, Guilford, CT (US); Tian Xu, Cambridge, MA (US); Henri Lichenstein, Guilford, CT (US)

(21) Appl. No.: 16/782,614

(22) Filed: Feb. 5, 2020

Related U.S. Application Data
(63) Continuation of application No. 15/678,304, filed on Aug. 16, 2017, now abandoned.
(60) Provisional application No. 62/376,970, filed on Aug. 19, 2016.

(51) Int. Cl. A61K 31/5377; A61K 31/713; A61K 38/21; A61K 45/06; A61K 31/724

(52) U.S. Cl. CPC A61K 31/5377; A61K 31/713; A61K 31/00; A61K 45/06; A61K 31/724; A61K 38/21

(57) ABSTRACT

The present invention relates to the use of PIKfyve inhibitors to treat Niemann-Pick disease type C, and related compositions and methods.

### FIELD OF THE INVENTION

The present invention relates to methods and compositions related to treating Niemann Pick C disease.

### BACKGROUND OF THE INVENTION

Niemann-Pick disease type C (NPC) one of a several diseases manifesting from cellular lysosomal storage defects which cause defects in metabolism. NPC is caused by genetic mutations in either the NPC1 or NPC2 gene. Individuals affected with NPC are unable to normally metabolize cholesterol and other lipids resulting in their accumulation in the liver, spleen, and brain. The disease is always fatal, with few patients living past forty years of age and most dying before age 10.

NPC1 mutant patient skin fibroblasts have been used as a robust cellular model of NPC1 disease (Chen 2010, Xu M et al 2012, Xu M et al 2014) and these cells display a number of NPC-related defects including intracellular accumulation of cholesterol other lipids. An additional therapeutic mechanism for lowering NPC1 mutant related cholesterol accumulation in the lysosome has been demonstrated through lysosome exocytosis in NPC cells (Chen 2010, Xu M et al 2012) and has also been proposed as a therapeutic mechanism for other Lysosome Storage diseases (Samie M et al 2014).

Apilimod, also referred to as STA-5326, hereinafter “apilimod”, is recognized as a potent transcriptional inhibitor of IL-12 and IL-23. See e.g., Wada et al. Blood 109 (2007): 1156-1164. IL-12 and IL-23 are inflammatory cytokines normally produced by immune cells, such as B-cells and macrophages, in response to antigenic stimulation. Autoimmune disorders and other disorders characterized by chronic inflammation are characterized in part by inappropriate production of these cytokines. In immune cells, the selective inhibition of IL-12/IL-23 transcription by apilimod was shown to be mediated by apilimod's direct binding to phosphatidylinositol-3-phosphate 5-kinase (PIKfyve). See e.g., Cai et al. Chemistry and Biol. 20 (2013):912-921. PIKfyve plays a role in Toll-like receptor signaling, which is important in innate immunity.

Based upon its activity as an immunomodulatory agent and a specific inhibitor of IL-12/IL-23, apilimod has been proposed as useful in treating autoimmune and inflammatory diseases and disorders. See e.g., U.S. Pat. Nos. 6,858,606 and 6,660,733 (describing a family of pyrimidine compounds, including apilimod, purportedly useful for treating diseases and disorders characterized by IL-12 or IL-23 overproduction, such as rheumatoid arthritis, sepsis, Crohn's disease, multiple sclerosis, psoriasis, or insulin dependent diabetes mellitus). Similarly, apilimod was suggested to be useful for treating certain cancers based upon its activity to inhibit c-Rel or IL-12/23, particularly in cancers where these cytokines were believed to play a role in promoting aberrant cell proliferation. See e.g., WO 2006/128129 and Baird et al., Frontiers in Oncology 3:1 (2013, respectively).

Apilimod was also found to inhibit the production of a range of osteogenic cytokines, including IL-12, IL-23, and TNFα, in addition to promoting the expression of inhibitors of osteoclast differentiation such as IL-10 and GM-CSF (Wada Y et al. PLoS One 2012; 7(4):e35069). WO 2005/000404 describes five pyrimidine compounds, including apilimod (Compound 12), as having inhibitory activity against osteoclast formation in an in vitro assay with an IC₅₀ of 15 nM.

The full range of apilimod's cellular activities continues to be elucidated and new uses for this compound, and PIKfyve inhibitors generally, have been discovered by the present inventors.

### SUMMARY OF THE INVENTION

The present invention is based, in part, on the discovery that a PIKfyve inhibitor, apilimod, is able to reduce intracellular cholesterol accumulation in NPC1 cells. NPC1 cells are characterized by an aberrant accumulation of cholesterol, mimicking the metabolic dysfunction characteristic of NPC.

Accordingly, the present disclosure provides methods and compositions related to the use of PIKfyve inhibitors for treating NPC. In embodiments, the disclosure provides a method of treating NPC by administering a PIKfyve inhibitor in amounts sufficient to inhibit the pathogenic accumulation of cholesterol that is characteristic of the disease.

In accordance with any of the foregoing embodiments, the PIKfyve inhibitor is selected from the group consisting of apilimod free base and salts thereof, including apilimod dimesylate, APY0201, and YM-201636. In embodiments, the PIKfyve inhibitor is apilimod dimesylate. In embodiments, the PIKfyve inhibitor is selected from apilimod free base or pharmaceutically acceptable salt, solvate, clathrate, hydrate, polymorph, prodrug, analog or derivative thereof. In embodiments, the PIKfyve inhibitor is an apilimod compound, an active metabolite of an apilimod compound, or a combination thereof.

In embodiments, the at least one PIKfyve inhibitor is combined with at least one additional active agent in a single dosage form. In embodiments, the at least one PIKfyve inhibitor is administered in a therapeutic regimen with at least one additional active agent, in the same or different dosage forms.

In embodiments, the PIKfyve inhibitor is administered orally, for example in the form of a tablet or capsule. In embodiments, the PIKfyve inhibitor is administered by injection or by addition to sterile infusion fluids for intravenous infusion and is in the form of a suitable sterile aqueous solution or dispersion.

In the methods described here, the at least one PIKfyve inhibitor can be administered by any suitable route and either in the same dosage form or in a different dosage form from the optional additional agent. In embodiments, administration is via an oral, intravenous, or subcutaneous route. In embodiments, administration is once daily, twice daily, or continuous for a period of time, for example one or several days or one or several weeks. Continuous administration may be performed, for example, by using slow release dosage form that is e.g., implanted in the subject, or via continuous infusion, for example using a pump device, which also may be implanted.

The invention also provides a pharmaceutical pack or kit comprising, in separate containers or in a single container, a unit dose of at least one PIKfyve inhibitor, and optionally at least one additional agent. In embodiments, the pharmaceutical pack or kit comprises at least one PIKfyve inhibitor that is an apilimod compound selected from apilimod free base, apilimod dimesylate, or a racemically pure enantiomer of an active metabolite of apilimod, and combinations thereof.

### BRIEF DESCRIPTION OF THE DRAWINGS

FIG. 1 depicts NPC1 mutant fibroblasts were treated with apilimod in dose response assay (4 hours treatment per day for a total of 3 days) to evaluate for the modulation of cholesterol accumulation visualized by the staining of filipin, a fluorescent Cholesterol binding dye. The Fluorescent microscopy images of the NPC1 cells showed a dose dependent [nanomolar (nM)] decrease of filipin stained cholesterol accumulations.

FIG. 2 depicts a biochemical assay measuring intracellular cholesterol was performed on lysates from a corresponding set of NPC1 cells treated with apilimod. The biochemical results confirm the apilimod mediated lowering of the cholesterol in the NPC1 cells observed by filipin staining.

FIG. 3: the rate of lysosome exocytosis can be monitored by the measurement of the release of lysosomal proteins such as Hexosaminidase Subunit Beta(HexB). In FIG. 3, NPC1 fibroblasts were treated with apilimod for 24 hours, the HexB release lysosome exocytosis assay was performed as previously described (Xu et al 2012). The rate of HexB release from NPC1 was increased by apilimod treatment providing evidence for an increased rate of lysosome exocytosis likely leading to cholesterol lowering in NPC1 cells.

### DETAILED DESCRIPTION OF THE INVENTION

The present disclosure provides compositions and methods related to the use of PIKfyve inhibitors for treating NPC in a subject in need thereof, preferably a human subject.

In embodiments, the invention provides methods for the treatment of NPC by administering to the subject a therapeutically effective amount of at least one PIKfyve inhibitor. In embodiments, the at least one PIKfyve inhibitor is selected from the group consisting of an apilimod compound, APY0201, and YM201636, or a pharmaceutically acceptable salt, solvate, clathrate, hydrate, polymorph, metabolite, prodrug, analog or derivative thereof.

In accordance with any of the embodiments described here, the at least one PIKfyve inhibitor is an apilimod compound. Apilimod is a selective inhibitor of PIKfyve (Cai et al. 2013 Chem. & Biol. 20:912-921). Based upon its ability to inhibit IL-12/23 production, apilimod has been suggested as useful for treating inflammatory and autoimmune diseases such as rheumatoid arthritis, sepsis, Crohn's disease, multiple sclerosis, psoriasis, or insulin dependent diabetes mellitus, and in cancers where these cytokines were believed to play a pro-proliferative role.

As used herein, the term “an apilimod compound” may refer to apilimod itself (free base), or may encompass pharmaceutically acceptable salts, solvates, clathrates, hydrates, polymorphs, prodrugs, analogs or derivatives of apilimod, as described below. The structure of apilimod is shown in Formula I:

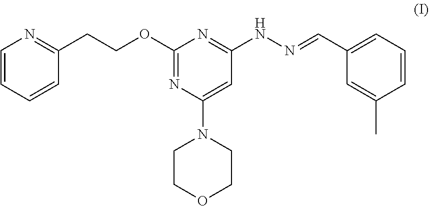 [SMILES: Cc1cccc(/C=N/Nc2cc(N3CCOCC3)nc(OCCc3ccccn3)n2)c1]

The chemical name of apilimod is 2-[2-Pyridin-2-yl)-ethoxy]-4-N′-(3-methyl-benzilidene)-hydrazino]-6-(morpholin-4-yl)-pyrimidine (IUPAC name: (E)-4-(6-(2-(3-methylbenzylidene)hydrazinyl)-2-(2-(pyridin-2-yl)ethoxy)pyrimidin-4-yl)morpholine), and the CAS number is 541550-19-0.

Apilimod can be prepared, for example, according to the methods described in U.S. Pat. Nos. 7,923,557, and 7,863,270, and WO 2006/128129.

In embodiments, the apilimod compound for use in the compositions and methods of the invention is the free base or dimesylate salt form, MW 610.7 (dimesylate salt); tPSA 83.1; pKa 5.39 (±0.03), 4.54 (±0.27); HBD 1. The apilimod dimesylate salt is highly water soluble (>25 mg/mL) and shows moderate permeability (>70% in rats). In embodiments, the apilimod compound for use in the compositions and methods of the invention is an active metabolite of apilimod. Six primary metabolites were identified in rat and human microsomal and hepatocyte stability studies. Human, rat, rabbit and dog studies showed a qualitatively similar metabolic profile. Tₘₐₓ generally occurred within 1 or 2 hours after the oral dose, consistent with the rapid elimination of this compound from the circulation. Reaction phenotyping studies indicated that CYP3A4 and to a lesser extent CYP1A2 and/or CYP2D6, contribute to metabolism. The primary metabolites are short-lived in circulation. Both apilimod free base and the dimesylate salt are highly bound (>99%) to rat, dog and human plasma proteins.

In embodiments, the at least one PIKfyve inhibitor is selected from APY0201 and YM-201636.

The chemical name of APY0201 is (E)-4-(5-(2-(3-methylbenzylidine)hydrazinlyl)-2-(pyridine-4-yl)pyrazolol[1,5-a]pyrimidin-7-yl)morpholine. APY0201 is a selective PIKfyve inhibitor (Hayakawa et al. 2014 Bioorg. Med. Chem. 22:3021-29). APY0201 directly interacts with the ATP-binding site of PIKfyve kinase, which leads to suppression of PI(3,5)P₂ synthesis, which in turn suppresses the production of IL-12/23.

The chemical name for YM201636 is 6-amino-N-(3-(4-morpholinopyrido[3′,2′:4,5]furo[3,2-d]pyrimidin-2-yl)phenyl)nicotinamide (CAS number is 371942-69-7). YM201636 is a selective inhibitor of PIKfyve (Jefferies et al. EMBO rep. 2008 9:164-170). It reversibly impairs endosomal trafficking in NIH3T3 cells, mimicking the effect produced by depleting PIKfyve with siRNA. YM201636 also blocks retroviral exit by budding from cells, apparently by interfering with the endosomal sorting complex required for transport (ESCRT) machinery. In adipocytes, YM-201636 also inhibits basal and insulin-activated 2-deoxyglucose uptake (IC₅₀=54 nM).

As used herein, the term “pharmaceutically acceptable salt,” is a salt formed from, for example, an acid and a basic group of an apilimod compound. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, besylate, gentisinate, fumarate, gluconate, glucaronate, saccharate, formate, benzoate, glutamate, methanesulfonate, ethanesulfonate, benzenesulfonate, p-toluenesulfonate, and pamoate (e.g., 1,1′-methylene-bis-(2-hydroxy-3-naphthoate)) salts. In a preferred embodiment, the salt of apilimod comprises methanesulfonate.

The term “pharmaceutically acceptable salt” also refers to a salt prepared from an apilimod compound having an acidic functional group, such as a carboxylic acid functional group, and a pharmaceutically acceptable inorganic or organic base.

The term “pharmaceutically acceptable salt” also refers to a salt prepared from an apilimod compound having a basic functional group, such as an amino functional group, and a pharmaceutically acceptable inorganic or organic acid.

The salts of the compounds described herein can be synthesized from the parent compound by conventional chemical methods such as methods described in Pharmaceutical Salts: Properties, Selection, and Use, P. Hemrich Stalil (Editor), Camille G. Wermuth (Editor), ISBN: 3-90639-026-8, August 2002. Generally, such salts can be prepared by reacting the parent compound with the appropriate acid in water or in an organic solvent, or in a mixture of the two.

One salt form of a compound described herein can be converted to the free base and optionally to another salt form by methods well known to the skilled person. For example, the free base can be formed by passing the salt solution through a column containing an amine stationary phase (e.g. a Strata-NH₂ column). Alternatively, a solution of the salt in water can be treated with sodium bicarbonate to decompose the salt and precipitate out the free base. The free base may then be combined with another acid using routine methods.

As used herein, the term “polymorph” means a solid crystalline form of a compound of the present invention. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing. For example, one polymorph might be more likely to form solvates or might be more difficult to filter or wash free of impurities than another due to, for example, the shape or size distribution of particles of it.

As used herein, the term “hydrate” means a compound of the present invention or a salt thereof, which further includes a stoichiometric or non-stoichiometric amount of water bound by non-covalent intermolecular forces.

As used herein, the term “clathrate” means a compound of the present invention or a salt thereof in the form of a crystal lattice that contains spaces (e.g., channels) that have a guest molecule (e.g., a solvent or water) trapped within.

As used herein, the term “prodrug” means a derivative of a compound described herein that can hydrolyze, oxidize, or otherwise react under biological conditions (in vitro or in vivo) to provide a compound of the invention. Prodrugs may only become active upon such reaction under biological conditions, or they may have activity in their unreacted forms. Examples of prodrugs contemplated in this invention include, but are not limited to, analogs or derivatives of a compound described herein that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of compounds of any one of the formulae disclosed herein that comprise —NO, —NO₂, —ONO, or —ONO₂ moieties. Prodrugs can typically be prepared using well-known methods, such as those described by Burger's Medicinal Chemistry and Drug Discovery (1995) 172-178, 949-982 (Manfred E. Wolff ed., 5th ed).

In addition, some of the compounds suitable for use in the methods of in this invention have one or more double bonds, or one or more asymmetric centers. Such compounds can occur as racemates, racemic mixtures, single enantiomers, individual diastereomers, diastereomeric mixtures, and cis- or trans- or E- or Z-double isomeric forms. All such isomeric forms of these compounds are expressly included in the present invention. The compounds of this invention can also be represented in multiple tautomeric forms, in such instances, the invention expressly includes all tautomeric forms of the compounds described herein (e.g., there may be a rapid equilibrium of multiple structural forms of a compound), the invention expressly includes all such reaction products). All such isomeric forms of such compounds are expressly included in the present invention. All crystal forms of the compounds described herein are expressly included in the present invention.

As used herein, the term “solvate” or “pharmaceutically acceptable solvate,” is a solvate formed from the association of one or more solvent molecules to one of the compounds disclosed herein. The term solvate includes hydrates (e.g., hemi-hydrate, mono-hydrate, dihydrate, trihydrate, tetrahydrate, and the like).

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound. As used herein, the term “derivative” refers to compounds that have a common core structure, and are substituted with various groups as described herein.

### Methods of Treatment

The disclosure provides methods for treating NPC using PIKfyve inhibitors and related compositions and methods.

In embodiments, the disclosure provides methods for treating NPC in a subject in need thereof by administering to the subject a therapeutically effective amount of at least one PIKfyve inhibitor.

In accordance with the embodiments described here, the therapeutically effective amount is the amount effective to inhibit the accumulation of cholesterol in target cells of the subject. In embodiments the target cells are selected from liver cells, spleen cells, and neural cells.

In accordance with any of the embodiments described here, the at least one PIKfyve inhibitor is selected from an apilimod compound, APY0201, YM-201636 or a pharmaceutically acceptable salt, solvate, clathrate, hydrate, polymorph, metabolite, prodrug, analog or derivative thereof. In embodiments, the PIKfyve inhibitor is apilimod dimesylate. In embodiments, the PIKfyve inhibitor is selected from apilimod free base or pharmaceutically acceptable salt, solvate, clathrate, hydrate, polymorph, prodrug, analog or derivative thereof. In embodiments, the PIKfyve inhibitor is an apilimod compound, an active metabolite of an apilimod compound, or a combination thereof.

The disclosure further provides the use of at least one PIKfyve inhibitor for the preparation of a medicament useful for the treatment of NPC as described herein.

In embodiments, the PIKfyve inhibitor is an apilimod compound and the therapeutically effective amount of the apilimod compound in humans is from about 0.001 mg/kg to about 1000 mg/kg, about 0.01 mg/kg to about 100 mg/kg, about 10 mg/kg to about 250 mg/kg, about 0.1 mg/kg to about 15 mg/kg; or any range in which the low end of the range is any amount between 0.001 mg/kg and 900 mg/kg and the upper end of the range is any amount between 0.1 mg/kg and 1000 mg/kg (e.g., 0.005 mg/kg and 200 mg/kg, 0.5 mg/kg and 20 mg/kg). Effective doses will also vary, as recognized by those skilled in the art. Effective doses will also vary, as recognized by those skilled in the art, depending on the diseases treated, route of administration, excipient usage, and the possibility of co-usage with other therapeutic treatments such as use of other agents. See e.g., U.S. Pat. No. 7,863,270, incorporated herein by reference.

In embodiments, an apilimod compound is administered to a human subject at a dosage regimen of 30-1000 mg/day (e.g., 30, 35, 40, 45, 50, 55, 60, 65, 70, 75, 80, 85, 90, 95, 100, 125, 150, 175, 200, 225, 250, 275, or 300 mg/day) for at least 1 week (e.g., 1, 2, 3, 4, 5, 6, 7, 8, 9, 10, 11, 12, 36, 48, or more weeks). Preferably, an apilimod compound is administered at a dosage regimen of 100-1000 mg/day for 4 or 16 weeks. Alternatively or subsequently, an apilimod compound is administered at a dosage regimen of 100 mg-300 mg twice a day for 8 weeks, or optionally, for 52 weeks. Alternatively or subsequently, an apilimod compound is administered at a dosage regimen of 50 mg-1000 mg twice a day for 8 weeks, or optionally, for 52 weeks.

In embodiments, the at least one PIKfyve inhibitor is administered once daily, from two to five times daily, up to two times or up to three times daily, or up to eight times daily. In embodiments, the at least one PIKfyve inhibitor is administered thrice daily, twice daily, once daily, fourteen days on (four times daily, thrice daily or twice daily, or once daily) and 7 days off in a 3-week cycle, up to five or seven days on (four times daily, thrice daily or twice daily, or once daily) and 14-16 days off in 3 week cycle, or once every two days, or once a week, or once every 2 weeks, or once every 3 weeks.

An effective amount of the apilimod compound can be administered once daily, from two to five times daily, up to two times or up to three times daily, or up to eight times daily. In one embodiment, the apilimod compound is administered thrice daily, twice daily, once daily, fourteen days on (four times daily, thrice daily or twice daily, or once daily) and 7 days off in a 3-week cycle, up to five or seven days on (four times daily, thrice daily or twice daily, or once daily) and 14-16 days off in 3 week cycle, or once every two days, or once a week, or once every 2 weeks, or once every 3 weeks.

The PIKfyve inhibitor (which may be referred to as “the inhibitor” for brevity) may be administered in a different dosage form, or in the same dosage form. Where the inhibitor is administered in separate dosage forms, they may be administered at the same time, or at different times.

A “subject” includes a mammal. The mammal can be e.g., any mammal, e.g., a human, primate, vertebrate, bird, mouse, rat, fowl, dog, cat, cow, horse, goat, camel, sheep or a pig. Preferably, the subject is a human. The term “patient” refers to a human subject.

As used herein, “treatment”, “treating” or “treat” describes the management and care of a patient for the purpose of combating a disease or disorder and includes the administration of a PIKfyve inhibitor, preferably an apilimod compound, to alleviate the symptoms or complications of the disease or disorder.

As used herein, “prevention”, “preventing” or “prevent” describes reducing or eliminating the onset of the symptoms or complications of a disease or disorder, includes the administration of a PIKfyve inhibitor, preferably an apilimod compound, to reduce the onset, development or recurrence of symptoms of the disease or disorder.

### Combination Therapies

The disclosure also provides methods comprising combination therapy. As used herein, “combination therapy” or “co-therapy” includes the administration of a therapeutically effective amount of a PIKfyve inhibitor, preferably an apilimod compound, with at least one additional active agent, as part of a specific treatment regimen intended to provide a beneficial effect from the co-action of the active agents in the regimen. In embodiments, the additional active agent may include a therapeutic agent conventionally used to treat NPC1. “Combination therapy” is not intended to encompass the administration of two or more therapeutic agents as part of separate monotherapy regimens that incidentally and arbitrarily result in a beneficial effect that was not intended or predicted.

In embodiments, the disclosure provides methods of treating NPC1 in a subject in need thereof using a combination therapy comprising a PIKfyve inhibitor, preferably an apilimod compound, and at least one additional therapeutic or non-therapeutic agent, or both. In embodiments, the additional therapeutic agent is selected from a modified cyclodextrin, such as VTS-270.

In embodiments, the methods include administration of at least one additional active agent that is a non-therapeutic agent, for which the beneficial effect of the combination may relate to the mitigation of toxicity, side effect, or adverse event associated with a therapeutically active agent in the combination. In embodiments, the non-therapeutic agent mitigates one or more side effects of an apilimod compound, the one or more side effects selected from any of nausea, vomiting, headache, dizziness, lightheadedness, drowsiness and stress. In one aspect of this embodiment, the non-therapeutic agent is an antagonist of a serotonin receptor, also known as 5-hydroxytryptamine receptors or 5-HT receptors. In one aspect, the non-therapeutic agent is an antagonist of a 5-HT3 or 5-HT1a receptor. In one aspect, the non-therapeutic agent is selected from the group consisting of ondansetron, granisetron, dolasetron and palonosetron. In another aspect, the non-therapeutic agent is selected from the group consisting of pindolol and risperidone.

In the context of combination therapy, administration of the PIKfyve inhibitor may be simultaneous with or sequential to the administration of the one or more additional active agents. In embodiments, administration of the different components of a combination therapy may be at different frequencies. The one or more additional agents may be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a compound of the present invention.

The one or more additional active agents can be formulated for co-administration with an apilimod composition in a single dosage form. The one or more additional active agents can be administered separately from the dosage form that comprises the PIKfyve inhibitor. When the additional active agent is administered separately from the PIKfyve inhibitor, it can be by the same or a different route of administration as the PIKfyve inhibitor.

Preferably, the administration of PIKfyve inhibitor in combination with one or more additional agents provides a synergistic response in the subject being treated. In this context, the term “synergistic” refers to the efficacy of the combination being more effective than the additive effects of either single therapy alone. The synergistic effect of a combination therapy according to the invention can permit the use of lower dosages and/or less frequent administration of at least one agent in the combination compared to its dose and/or frequency outside of the combination. Additional beneficial effects of the combination can be manifested in the avoidance or reduction of adverse or unwanted side effects associated with the use of either therapy in the combination alone (also referred to as monotherapy).

In certain embodiments the at least one PIKfyve inhibitor is provided in a single dosage form in combination with one or more additional therapeutic agents. In another embodiment, the apilimod compound is provided in combination with one or more additional PIKfyve inhibitors, for example APY0201 and YM201636. Where more than one therapeutic agent is present in a single dosage form, the therapeutically effective amount is based upon the total amount of therapeutic agents in the dosage form.

In one embodiment the at least one PIKfyve inhibitor is provided in a separate dosage form from the one or more additional therapeutic agents. Separate dosage forms are desirable, for example, in the context of a combination therapy in which the therapeutic regimen calls for administration of different therapeutic agents at different frequencies or under different conditions, or via different routes.

In one embodiment, administration of the at least one PIKfyve inhibitor as described herein is accomplished via an oral dosage form suitable for oral administration. In another embodiment administration is by an indwelling catheter, a pump, such as an osmotic minipump, or a sustained release composition that is, for example, implanted in the subject.

### Pharmaceutical Compositions and Formulations

The disclosure also provides pharmaceutical compositions comprising an effective amount of at least one PIKfyve inhibitor and at least one pharmaceutically acceptable excipient or carrier, wherein the effective amount is as described above in connection with the methods of the invention.

In embodiments, the PIKfyve inhibitor is selected from one or more of an apilimod compound, APY0201, YM-201636, and pharmaceutically acceptable salts, solvates, clathrates, hydrates, polymorphs, metabolites, prodrugs, analogs and derivatives thereof. In one embodiment, the PIKfyve inhibitor is an apilimod compound.

In embodiments, the at least one PIKfyve inhibitor is further combined with at least one additional therapeutic agent in a single dosage form. Suitable additional therapeutic agents are described in detail supra.

The term “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. Examples of pharmaceutically acceptable excipients include, without limitation, sterile liquids, water, buffered saline, ethanol, polyol (for example, glycerol, propylene glycol, liquid polyethylene glycol and the like), oils, detergents, suspending agents, carbohydrates (e.g., glucose, lactose, sucrose or dextran), antioxidants (e.g., ascorbic acid or glutathione), chelating agents, low molecular weight proteins, or suitable mixtures thereof.

A pharmaceutical composition can be provided in bulk or in dosage unit form. It is especially advantageous to formulate pharmaceutical compositions in dosage unit form for ease of administration and uniformity of dosage. The term “dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved. A dosage unit form can be an ampoule, a vial, a suppository, a dragee, a tablet, a capsule, an IV bag, or a single pump on an aerosol inhaler.

In therapeutic applications, the dosages vary depending on the agent, the age, weight, and clinical condition of the recipient patient, and the experience and judgment of the clinician or practitioner administering the therapy, among other factors affecting the selected dosage. Generally, the dose should be a therapeutically effective amount. Dosages can be provided in mg/kg/day units of measurement (which dose may be adjusted for the patient's weight in kg, body surface area in m², and age in years). Exemplary doses and dosages regimens for the compositions in methods of treating NPC1 are described above.

A dose may be provided in unit dosage form. For example, the unit dosage form can comprise 1 nanogram to 2 milligrams, or 0.1 milligrams to 2 grams; or from 10 milligrams to 1 gram, or from 50 milligrams to 500 milligrams or from 1 microgram to 20 milligrams; or from 1 microgram to 10 milligrams; or from 0.1 milligrams to 2 milligrams.

The pharmaceutical compositions can take any suitable form (e.g, liquids, aerosols, solutions, inhalants, mists, sprays; or solids, powders, ointments, pastes, creams, lotions, gels, patches and the like) for administration by any desired route (e.g, pulmonary, inhalation, intranasal, oral, buccal, sublingual, parenteral, subcutaneous, intravenous, intramuscular, intraperitoneal, intrapleural, intrathecal, transdermal, transmucosal, rectal, and the like). For example, a pharmaceutical composition of the invention may be in the form of an aqueous solution or powder for aerosol administration by inhalation or insufflation (either through the mouth or the nose), in the form of a tablet or capsule for oral administration; in the form of a sterile aqueous solution or dispersion suitable for administration by either direct injection or by addition to sterile infusion fluids for intravenous infusion; or in the form of a lotion, cream, foam, patch, suspension, solution, or suppository for transdermal or transmucosal administration.

A pharmaceutical composition can be in the form of an orally acceptable dosage form including, but not limited to, capsules, tablets, buccal forms, troches, lozenges, and oral liquids in the form of emulsions, aqueous suspensions, dispersions or solutions. Capsules may contain mixtures of a compound of the present invention with inert fillers and/or diluents such as the pharmaceutically acceptable starches (e.g., corn, potato or tapioca starch), sugars, artificial sweetening agents, powdered celluloses, such as crystalline and microcrystalline celluloses, flours, gelatins, gums, etc. In the case of tablets for oral use, carriers which are commonly used include lactose and corn starch. Lubricating agents, such as magnesium stearate, can also be added. For oral administration in a capsule form, useful diluents include lactose and dried corn starch. When aqueous suspensions and/or emulsions are administered orally, the compound of the present invention may be suspended or dissolved in an oily phase is combined with emulsifying and/or suspending agents. If desired, certain sweetening and/or flavoring and/or coloring agents may be added.

A pharmaceutical composition can be in the form of a tablet. The tablet can comprise a unit dosage of a compound of the present invention together with an inert diluent or carrier such as a sugar or sugar alcohol, for example lactose, sucrose, sorbitol or mannitol. The tablet can further comprise a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. The tablet can further comprise binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures.

The tablet can be a coated tablet. The coating can be a protective film coating (e.g. a wax or varnish) or a coating designed to control the release of the active agent, for example a delayed release (release of the active after a predetermined lag time following ingestion) or release at a particular location in the gastrointestinal tract. The latter can be achieved, for example, using enteric film coatings such as those sold under the brand name Eudragit®.

Tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate, and triethanolamine.

A pharmaceutical composition can be in the form of a hard or soft gelatin capsule. In accordance with this formulation, the compound of the present invention may be in a solid, semi-solid, or liquid form.

A pharmaceutical composition can be in the form of a sterile aqueous solution or dispersion suitable for parenteral administration. The term parenteral as used herein includes subcutaneous, intracutaneous, intravenous, intramuscular, intra-articular, intraarterial, intrasynovial, intrasternal, intrathecal, intralesional and intracranial injection or infusion techniques.

A pharmaceutical composition can be in the form of a sterile aqueous solution or dispersion suitable for administration by either direct injection or by addition to sterile infusion fluids for intravenous infusion, and comprises a solvent or dispersion medium containing, water, ethanol, a polyol (e.g., glycerol, propylene glycol and liquid polyethylene glycol), suitable mixtures thereof, or one or more vegetable oils. Solutions or suspensions of the compound of the present invention as a free base or pharmacologically acceptable salt can be prepared in water suitably mixed with a surfactant. Examples of suitable surfactants are given below. Dispersions can also be prepared, for example, in glycerol, liquid polyethylene glycols and mixtures of the same in oils.

The pharmaceutical compositions for use in the methods of the present invention can further comprise one or more additives in addition to any carrier or diluent (such as lactose or mannitol) that is present in the formulation. The one or more additives can comprise or consist of one or more surfactants. Surfactants typically have one or more long aliphatic chains such as fatty acids which enables them to insert directly into the lipid structures of cells to enhance drug penetration and absorption. An empirical parameter commonly used to characterize the relative hydrophilicity and hydrophobicity of surfactants is the hydrophilic-lipophilic balance (“HLB” value). Surfactants with lower HLB values are more hydrophobic, and have greater solubility in oils, while surfactants with higher HLB values are more hydrophilic, and have greater solubility in aqueous solutions. Thus, hydrophilic surfactants are generally considered to be those compounds having an HLB value greater than about 10, and hydrophobic surfactants are generally those having an HLB value less than about 10. However, these HLB values are merely a guide since for many surfactants the HLB values can differ by as much as about 8 HLB units, depending upon the empirical method chosen to determine the HLB value.

Among the surfactants for use in the compositions of the invention are polyethylene glycol (PEG)-fatty acids and PEG-fatty acid mono and diesters, PEG glycerol esters, alcohol-oil transesterification products, polyglyceryl fatty acids, propylene glycol fatty acid esters, sterol and sterol derivatives, polyethylene glycol sorbitan fatty acid esters, polyethylene glycol alkyl ethers, sugar and its derivatives, polyethylene glycol alkyl phenols, polyoxyethylene-polyoxypropylene (POE-POP) block copolymers, sorbitan fatty acid esters, ionic surfactants, fat-soluble vitamins and their salts, water-soluble vitamins and their amphiphilic derivatives, amino acids and their salts, and organic acids and their esters and anhydrides.

The present invention also provides packaging and kits comprising pharmaceutical compositions for use in the methods of the present invention. The kit can comprise one or more containers selected from the group consisting of a bottle, a vial, an ampoule, a blister pack, and a syringe. The kit can further include one or more of instructions for use in treating and/or preventing a disease, condition or disorder of the present invention, one or more syringes, one or more applicators, or a sterile solution suitable for reconstituting a pharmaceutical composition of the present invention.

All percentages and ratios used herein, unless otherwise indicated, are by weight. Other features and advantages of the present invention are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

### EXAMPLES

### Example 1: Apilimod-Induced Reduction in Intracellular Cholesterol in NPC1 Fibroblasts

The target of apilimod is the lipid kinase phosphatidylinositol-3-phosphate 5-kinase (PIKfyve), which phosphorylates endosomal PI3P to generate the phosphoinositide PI(3,5)P2 (Boyle W J et al., Nature. 2003 May 15; 423(6937):337-42). Loss of PI(3,5)P2 through PIKfyve inhibition is associated with extensive endomembrane vacuolization and disruption of endolysosomal trafficking.

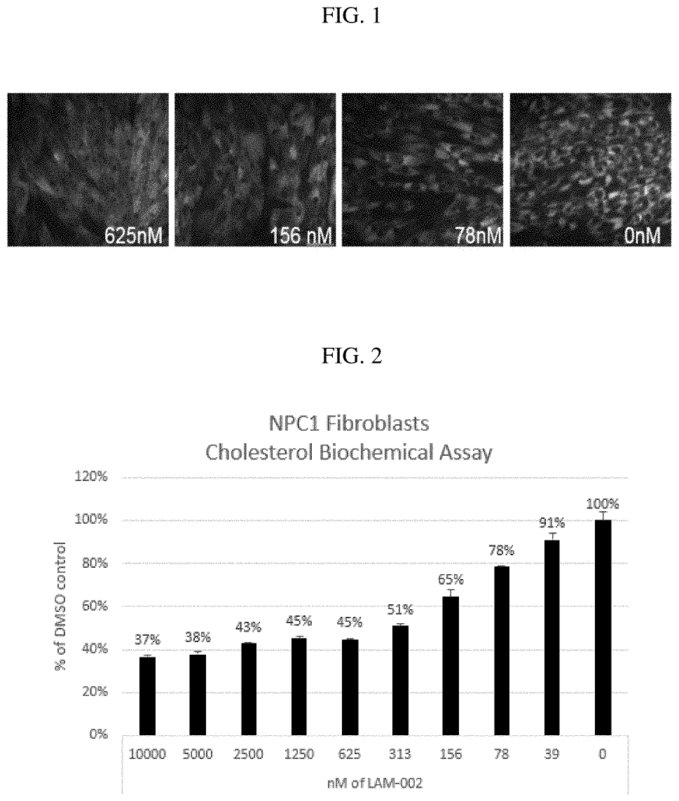

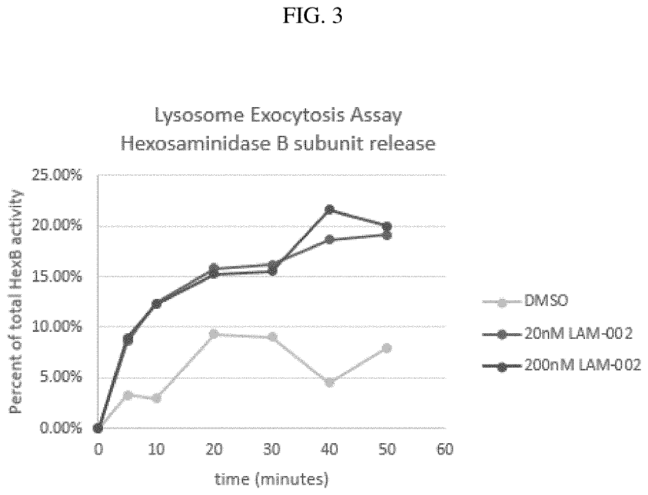

Surprisingly, treatment of NPC1 fibroblasts, a cellular model of NPC1 disease (Chen 2010, Xu M et al 2012, Xu M et al 2014), with apilimod reduced intracellular cholesterol accumulation in a dose-dependent manner. FIG. 1 shows NPC1 fibroblasts treated with apilimod dimesylate in dose response assay (4 hours treatment per day for total of 3 days) to evaluate and cholesterol accumulation visualized by filipin staining (filipin is a fluorescent cholesterol binding dye). Shown in the figure are fluorescence microscopy images of the NPC1 cells treated with 0, 78, 156, and 625 nM apilimod. The results show a dose dependent decrease of filipin fluorescence with apilimod treatment. FIG. 2 shows a biochemical assay measuring intracellular cholesterol performed on lysates from NPC1 cells treated with apilimod dimesylate (0 to 10,000 nM). The biochemical results confirm that apilimod dimesylate treatments results in lower intracellular cholesterol in NPC1 fibroblasts. In FIG. 3, apilimod treatment of NPC1 fibroblasts can enhance the rate of lysosome exocytosis as one of the mechanisms for cells to clear lysosome storage of cholesterol and lipid accumulations in NPC.

1-16. (canceled)

17. A method for decreasing cholesterol accumulation in a target cell of a subject in need thereof, the method comprising administering to the subject at least one PIKfyve inhibitor selected from the group consisting of apilimod or a pharmaceutically acceptable salt thereof, APY0201 ((E)-4-(5-(2-(3-methylbenzylidine)hydrazinyl)-2-(pyridin-4-yl)pyrazolo[1,5-a]pyrimidin-7-yl)morpholine) and YM-201636 (6-amino-N-(3-(4-morpholinopyrido[3′,2′:4,5]furo[3,2-d]pyrimidin-2-yl)phenyl)nicotinamide).

18. The method of claim 17, wherein the target cell is selected from a liver cell, a spleen cell, and a neural cell.

19. The method of claim 18, wherein the target cell is a liver or spleen cell.

20. The method of claim 17, wherein the subject is human.

21. The method of claim 20, wherein the subject in need is a subject having Niemann-Pick disease type C.

22. The method of claim 21, wherein the PIKfyve inhibitor is apilimod or a pharmaceutically acceptable salt thereof.

23. The method of claim 22, wherein the pharmaceutically acceptable salt of apilimod is selected from sulfate, citrate, oxalate, chloride, bromide, iodide, nitrate, bisulfate, phosphate, acid, acid phosphate, isonicotinate, lactate, salicylate, acid citrate, tartrate, oleate, tannate, pantothenate, bitartrate, ascorbate, succinate, maleate, besylate, gentisinate, fumarate, gluconate, glucaronate, saccharate, formate, benzoate, glutamate, methanesulfonate, ethanesulfonate, benzenesulfonate, p-toluenesulfonate, and pamoate.

24. The method of claim 23, wherein the pharmaceutically acceptable salt of apilimod is selected from the group of selected from a chloride, methanesulfonate, fumarate, lactate, maleate, pamoate, phosphate, and tartrate.

25. The method of claim 24, wherein the pharmaceutically acceptable salt of apilimod is methanesulfonate.
